(19) United States
(12) Patent Application Publication
(10) Pub. No.: US 2016/0045154 A1
(43) Pub. Date: Feb. 18, 2016

(54) SLEEP APNEA

(71) Applicant: ORIDION MEDICAL 1987 LTD., Jerusalem (IL)

(72) Inventors: Paul Stanley Addison, Edinburgh (GB); Joshua Lewis Colman, Jerusalem (IL); Mark Golberg, Ashdod (IL); Konstantin Goulitski, Holon (IL)

(21) Appl. No.: 14/458,264

(22) Filed: Aug. 13, 2014

(51) Int. Cl. A61B 5/00; A61B 5/1455; A61B 5/0402; A61M 21/02; A61B 5/02; A61B 5/026; A61M 16/00; A61B 5/0205; A61B 5/0476

(52) U.S. Cl. CPC A61B 5/4818; A61B 5/0205; A61B 5/7275; A61B 5/7267; A61B 5/14552; A61B 5/0402; A61B 5/0476; A61B 5/7278; A61B 5/02028; A61B 5/026; A61M 16/0069; A61M 21/02; A61B 5/7425; A61B 5/4836; A61B 5/0836

(57) ABSTRACT

Provided are systems and method for prediction and mitigation of apnea of a subject, based on measurements of various physiological parameters of the subject.

### TECHNICAL FIELD

The present disclosure generally relates to medical monitoring systems and methods of using the same for prediction and prevention of sleep apnea.

### BACKGROUND

Sleep Apnea is a type of sleep disorder characterized by pauses in breathing or instances of shallow or infrequent breathing during sleep. Each pause in breathing, called an apnea, can last from number of seconds to minutes, and may occur 2 to more than 30 times per hour. When breathing is paused, carbon dioxide is building up the bloodstream and consequently, the brain is signaled to wake the person sleeping and breathe in air. Breathing normally will restore oxygen levels and the subject will fall asleep again. Sleep apnea may often be diagnosed with an overnight sleep test called a polysomnogram. In general, three types of sleep apnea may be recognized: central (CSA), obstructive (OSA), and complex or mixed sleep apnea. In CSA, breathing is interrupted by a lack of respiratory effort and in OSA, breathing is interrupted by a physical block to airflow despite respiratory effort. Apnea symptoms may be present for many years without identification, during which time the subject may become conditioned to daytime sleepiness and fatigue, as well as increased risk of developing related conditions, such as, diabetes, depression, and hypertension.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other advantages or improvements.

According to some embodiments, there are provided methods and systems for detection, prediction and/or anticipation of an apnea event and mitigation thereof, in a subject having apnea. In some embodiments, the systems and methods disclosed herein provide mitigation without affecting the awareness of the subject, i.e., without wakening the subject while mitigating/preventing the predicted apnea event. In some embodiments, the systems and methods disclosed herein provide a real time identification/prediction of an apnea event and allow the real time mitigation thereof.

In some embodiments, the methods provided herein take use of measurements or related information obtained from one or more medical monitoring devices. In some embodiments, the medical monitoring devices may be selected from, but not limited to: capnograph, pulse oximeter and heart monitor. In some embodiments, the measurements or related information obtained from the medical monitoring devices are various physiological parameters, such as, for example, but not limited to: breath related parameters (as determined, for example, based on CO₂ measurements in exhaled breath), heart rate related parameters, blood related parameters, brain activity related parameters, and the like, or combinations thereof.

According to some embodiments, there is provided system for prediction of an apnea event, the system comprising: a medical monitoring device configured to detect a breath related parameter of the subject; and a stimulating device configured to mitigate said apnea event.

According to some embodiments there is provided a system for predicting and mitigating an apnea event, the system including one or more medical devices configured to measure one or more physiological parameters of a subject; a processing unit capable of integrating the measured physiological parameters to predict an upcoming apnea event, based on the parameters; and an intervention/stimulating unit/device configured to mitigate the apnea event.

In some embodiments, the processing unit may be configured to determine a patient specific pre-apneic pattern in the one or more physiological parameters by applying a learning algorithm to the measured one or more physiological parameters. In some embodiments, predicting the apnea event may include detecting the determined patient specific pre-apneic pattern in the one or more measured physiological parameters.

In some embodiments, the medical monitoring devices may be selected from: a capnograph and a pulse oximeter. In some embodiments, the processing unit may integrate more than one discrete physiological parameter. In some embodiments, the processing unit may integrate at least two discrete physiological parameters. In some embodiments, the processing unit may integrate at least three discrete physiological parameters and/or their patterns. In some embodiments, at least one of the measured physiological parameters may be expired CO₂. In some embodiments, at least one of the measured physiological parameters may be a SpO₂ related parameter.

In some embodiments, the measured parameters may be further manipulated and/or processed prior to or simultaneously while being integrated by the processing unit.

Surprisingly, the methods and systems disclosed herein, which can accurately predict apnea event, based on measurement of various health related parameters (such as CO₂ in breath (expired CO₂), SpO₂ measurements, air flow measurements, and the like), and/or data derived therefrom, provide a more accurate and reliable means for determining, evaluating and/or predicting apnea events, which is detected/predicted based on changes in the measured parameters. Thus, the systems and methods disclosed herein advantageously provide an efficient, accurate, non-invasive and continuous means for predicting and/or controlling apnea event of a subject. The methods and systems disclosed herein advantageously further provide mitigation of the predicted apnea event, without affecting the awareness of the subject, i.e., without wakening the subject, thereby allowing the subject to sleep without interruption to prevent the severe side effects associated with apnea. The methods and systems disclosed herein advantageously may further allow the accurate prediction of central apnea and mitigation thereof, in particular for subjects recovering from surgery. Such subjects have high levels of sedatives in their system, which makes them susceptible to respiratory depression, which may result in fatal consequences.

According to some embodiments, there is provided a system for predicting and mitigating sleep apnea event in a subject, the system comprising: one or more medical monitoring devices configured to measure one or more physiological parameters and/or their patterns of the subject; a processing unit configured to integrate the one or more physiological parameters of the subject, to predict an apnea event, based on the measured parameters; and a stimulating unit configured to provide a stimulation to the subject, prior to the apnea event, thereby mitigating the apnea event.

According to some embodiments, the medical monitoring device may include a capnograph, a pulse oximeter, a breath flow sensor, an Electrocardiogram (ECG), a Brain activity monitoring device, or combinations thereof.

According to some embodiments, the physiological parameters of the subject may include: breath related parameters, heart related parameters, blood related parameters, brain electrical activity, parameters derived therefrom, or combinations thereof.

According to some embodiments, the breath related parameters may include such parameters as, but not limited to: airflow, respiration rate, respiration effort, expired CO₂, or combinations thereof. Various expired CO₂ related parameters may include such parameters as, but not limited to: CO₂ concentration in breath, EtCO₂, CO₂ waveform, CO₂ volume in expired breath, Respiration rate, respiration effort, data derived therefrom, and combinations thereof.

According to some embodiments, the heart related parameters may include heart rate, amplitude of cardiac pulses, Percent Modulation (PMod) of the cardiac pulses, or combinations thereof.

According to some embodiments, the blood related parameters may include blood pressure, SpO₂, or both.

According to some embodiments, stimulating unit does not induce wakening of the subject. In further embodiments, the stimulating unit may include a gas emitting unit, a gas dispenser, a tactile unit, an audible unit, or combinations thereof. In some embodiments, the gas administered to the subject is CO₂.

According to some embodiments, the system may further include a display unit configured to display one or more of: one or more of the measured parameters, parameters derived from the measured parameters, an indication for a predicated apnea event, an indication of the activation of the stimulating unit, or combinations thereof.

According to some embodiments, there is provided a method for predicting and mitigating a sleep apnea event in a subject, the method comprising:
a) obtaining a measurement of one or more physiological parameters of the subject;
b) predicting an apnea event based on the measured parameters of the subject; and
c) issuing a stimulation prior to the apnea event to mitigate said apnea event.

Certain embodiments of the present disclosure may include some, all, or none of the above advantages. One or more other technical advantages may be readily apparent to those skilled in the art from the figures, descriptions, and claims included herein. Moreover, while specific advantages have been enumerated above, various embodiments may include all, some, or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES:

FIG. 1—a schematic presentation of a system for predicting and mitigating apnea event of a subject, according to some embodiments;

FIG. 2—graphs depicting changes in various physiological parameters of a subject, for the prediction of an apnea event, according to some embodiments; and

FIG. 3—a schematic block diagram of steps in a method for predicting and mitigating sleep apnea, according to some embodiments.

### DETAILED DESCRIPTION

In the following description, various aspects of the disclosure will be described. For the purpose of explanation, specific configurations and details are set forth in order to provide a thorough understanding of the disclosure. However, it will also be apparent to one skilled in the art that the disclosure may be practiced without specific details being presented herein. Furthermore, well-known features may be omitted or simplified in order not to obscure the disclosure.

As referred to herein, the terms “user”, “medical user”, “health care provider” and “health care professional” may interchangeably be used. The terms may include any health care provider who may treat and/or attend to a patient. A user may include, for example, a nurse, respiratory therapist, physician, anesthesiologist, and the like. In some cases, a user may also include a patient.

As referred to herein, the terms “device”, “monitoring device” and “medical device” may interchangeably be used. Exemplary monitoring devices include such devices as, but not limited to: Capnograph, pulse oximeter, breath flowmeter or sensor, Electrocardiograph (ECG), skin humidity measuring device, thermometer, Brain electrical activity monitoring device (such as BIS), and the like.

As referred to herein, the term “physiological parameter” is directed to a health related parameter of the subject. The health related parameter may be directly and/or indirectly measured, detected and/or derived from a measurement of a medical monitoring device, for example, via an appropriate sensor. In some embodiments, the health related parameter may include such parameters as, but not limited to: breath related parameters (such as, for example, CO₂ related parameters, EtCO₂, O₂ related parameters, breath rate, breath cycle, respiration rate, breath flow and the like); heart related parameters (such as, pulse rate); blood related parameters (such as, blood pressure); temperature; skin related parameters (such as, skin humidity, skin conductance, skin capacitance, and the like); Brain related parameters (such as, brain activity); and the like; or combinations thereof.

As referred to herein, the terms “patient” and subject” may interchangeably be used and may relate to a subject being monitored by any monitoring device for any physical-condition related parameter and/or health related parameter.

As referred to herein, the term “waveform” is directed to a recurring graphic shape which may be realized by measuring a physiological parameter of a subject over time, such as, for example, concentration of CO₂ in breath, follow rate of breath, electrocardiogram (ECG), plethysmograph, and the like. In some embodiments, a waveform is a medically, time resolved waveform. A waveform may have various characteristic parameters/features/factors that may be derived from the shape, dimension, rate or frequency, reoccurrences, and the like, and combinations thereof.

As referred to herein, the term “pattern(s)” relates to any identified/determined pattern over time, which is recurring known or known that may be produced when graphically displaying any of the waveforms or waveforms related factors/parameters. In some embodiments, a pattern may be predefined. In some embodiments, a pattern may be determined if it is clearly repeating itself for a given number of times over a given period of time.

As referred to herein, the terms ordinary, normal, typical, standard and common may interchangeably be used.

As referred to herein, the term “EtCO₂” relates to End tidal CO₂. The CO₂ is exhaled out of the body and the concentration of the exhaled CO₂, also known as end tidal CO₂ (EtCO₂) is an approximate estimation of the alveolar CO₂ pressure and thus of the arterial levels of CO₂. The values of EtCO2 may be measured in units of pressure, such as, for example, mmHg.

As referred to herein, the terms “VCap” or “volumetric capnography” may be interchangeably used. The terms refer to the volume of CO₂ in an expired breath.

As referred to herein, the term “breath cycle” includes the stages of exhalation and inhalation. The breath cycle may be derived from a CO₂ waveform which depicts the change in expired CO₂ concentration over time, (EtCO₂). During a breath cycle, the levels of CO₂ initially increase as a result of CO₂ release from the airways, from what is known as the “dead space”, which is the space in which no gas exchange takes place. Then, the CO₂ rapidly reaches a plateau at high levels of CO₂, which corresponds to the release of CO₂ from the lungs, in the exhalation phase. A rapid decline in exhaled CO₂ proceeds the inhalation phase, characterized by absence/minute levels of CO₂.

As referred to herein, the term “Respiration Rate” (RR) may be defined as the number of breaths taken in a minute, and it may change under various physiological and medical conditions.

As referred to herein, the terms “Heart Rate” (HR) and “pulse rate” may interchangeably be used and may relate to the number of heart pulses (beats) in a minute. Pulse rate is usually considered to be a combination of left ventricular stroke volume, ejection velocity, the relative compliance and capacity of the arterial system, and the pressure waves that result from the antegrade flow of blood and reflections of the arterial pressure pulse returning from the peripheral circulation, and some or all of which may be effected by CO₂.

In some embodiments, the terms “calculated” and “computed” may interchangeably be used.

The terms “apnea”, “sleep apnea”, and “apnea event” may interchangeably be used. The terms refer to sleep apnea, in which pauses in breathing or instances of shallow or infrequent breathing during sleep are present. Each pause in breathing, called an apnea, can last from number of seconds to minutes, and may occur 2 to more than 30 times per hour. The terms relate to all types of sleep apnea, including central sleep apnea (CSA), obstructive sleep apnea (OSA), and complex or mixed sleep apnea.

The terms “desaturation event” or “desat” and “desaturation” may interchangeably be used. The term refers to a drop (e.g. a 3% drop or more) in the oxygen levels of the patient. Desaturation events may occur during apnea or hypopnea events while asleep.

The term “mitigation” as used herein is directed to encompass the attenuation, prevention, moderation, easing, alleviation, extenuation and/or vindication of an apnea event. In some embodiments, the mitigation of an apnea event is performed before the apnea event occurred. In some embodiments, the mitigation of the apnea event is performed at the onset of the apnea event. In some embodiments, the mitigation of the apnea event is performed together with the apnea event. In some embodiments, the mitigation of an apnea event does not induce full awakening of the subject having or is predicted to have an apnea event. According to some embodiments, mitigating the apnea event may include diminishing the arousal and/or awakening of the patient.

The term “arousals”, as used herein, may refer to an interruptions of sleep lasting less than 15 seconds. The arousal may occur spontaneously or as a result of sleep-disordered breathing or other sleep disorders. The sleep following an arousal may often be lighter. According to some embodiments, an arousal may occur without the subject being aware thereof.

The terms “awakening”, “wakening”, “awake”, and “wake' may interchangeably be used. The terms relate to an interruption of sleep lasting more than 15 seconds. According to some embodiments, a subject awaked may typically be aware thereof.

Capnography is a non-invasive monitoring method used to continuously measure CO₂ concentration in exhaled breath. The CO₂, which is a constant metabolism product of the cells, is exhaled out of the body, and the concentration of the exhaled CO₂, also known as end tidal CO₂ (EtCO₂), is an approximate estimation of the arterial levels of CO₂. Capnograph (or capnometer) is a medical monitoring device that may be used for measuring the carbon dioxide (CO₂) content in inspired and expired air of a subject. It is a non-invasive device that measures the concentrations of respired gases.

Breath flow sensors are medical monitoring devices that may be used for measuring breath flow patterns. The breath flow sensor may be a spirometer used for assessing lung function, specifically the amount (volume) and/or speed (flow) of air that can be inhaled and exhaled.

Spirometry may be used in assessing conditions such as asthma, pulmonary fibrosis, cystic fibrosis, and COPD.Pulse oximeter is a type of oximeter, which is a medical monitoring device that may be used to determine oxygen saturation of the blood. Pulse oximeter may indirectly be used to measure the oxygen (O₂) saturation concentration and changes in blood volume in the skin (i.e., act as a photoplethysmograph). Pulse oximeter may also be used to measure the pulse rate of a subject. The term SpO₂ relates to the saturation of peripheral oxygen. It is a measurement of the amount of oxygen attached to the hemoglobin in red blood cells in the circulatory system. SpO₂ values are generally given as a percentage. SpO₂ may be monitored and measured by a Pulse Oximeter.

According to some embodiments, there are provided methods and systems for predicting and/or mitigating apnea event of a subject, without arousing/wakening the subject.

In some embodiments, the systems and methods provided herein take use of measurements of physiological parameters and/or information/data derived therefrom, obtained from one or more medical monitoring devices. In some embodiments, the medical monitoring devices may be selected from, but not limited to: capnograph, photoplethysmograph (such as, pulse oximeter), ECG, BIS, EEG, and the like. In some embodiments, the physiological parameters, or data derived therefrom, are such parameters as, for example, but not limited to: breath related parameters (as determined, for example, based on expired CO₂ (measurements of CO₂ in exhaled breath), heart related parameters (such as heart rate), blood related parameters (such as, blood pressure, oxygen saturation), brain activity related parameters (such as brain electrical activity), body temperature, and the like, or combinations thereof.

According to some embodiments there is provided a system for predicting and/or mitigating apnea event of a subject, the system may include one or more medical monitoring devices configured to obtain, acquire or measure one or more physiological parameters of a subject; a processing unit capable of integrating the measured physiological parameters to predict an apnea event of the subject based on the measured parameters, and optionally a stimulating unit configured to provide a stimulation to the subject to mitigate the apnea event, preferably without wakening the subject. In some embodiments, the system comprises a combination of at least two medical monitoring devices. In some embodiments, the system may include a combination of at least three medical monitoring devices. In some embodiments, the medical monitoring devices may be selected from, but not limited to: a capnograph, a pulse oximeter, heart monitor, brain activity monitor, airflow meter, and the like, or any combination thereof. In some embodiments, the processing unit may integrate more than one discrete physiological parameter. In some embodiments, the processing unit may integrate at least two discrete physiological parameters. In some embodiments, the processing unit may integrate at least three discrete physiological parameters. In some embodiments, the measured physiological parameters may include such parameters as, but not limited to: breath related parameters (such as, CO₂ measurements in exhaled breath, airflow, respiration rate, respiration effort, and the like), heart rate related parameters (such as heart rate), blood related parameters (such as, blood pressure, oxygen saturation, venous return flow from peripherals, and the like), brain activity related parameters (such as brain electrical activity), body temperature, parameters derived therefrom, or combinations thereof.

In some embodiments, the measured parameters may be further manipulated and/or processed to generate data related to the measurements, prior to or concomitantly while being integrated by the processing unit. For example, in some embodiments, various CO₂ related parameters may be derived from the measurement of expired CO₂, such as, for example but not limited to: CO₂ waveform and parameters related thereto (such as, for example, but not limited to: EtCO2, changes in EtCO₂, a slope of the increase in the CO₂ concentration, a change in a slope of the increase in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. For example, in some embodiments, various parameters (data) derived from pulse oximetry measurements may be obtained, such as, heart rate (pulse rate), respiration effort, amplitude of cardiac pulses, as determined based on the photoplethysmogram signal; modulation of the amplitude of cardiac pulses, percent modulation (PMod) of the signal, changes in the PMod, change in venous return flow from the peripherals, and the like, or combinations thereof.

According to some embodiments, a lengthening in the fall of the CO₂ waveform may be indicative of an imminent apnea event. According to some embodiments, reduction in the value of flow parameters may be indicative of an upcoming apnea event. According to some embodiments, a reduction in the volume of CO₂ in an expired breath may be in indicative of an apnea event. According to some embodiments, a desaturation event may be indicative of an apnea event.

In some embodiments the measured parameters and/or the data related thereto may be used to predict an apnea event. In some embodiments, a combination of measured parameters and/or data related thereto may be used to predict an apnea event. In some embodiments, the parameters or data related thereto may be obtained from one or more medical monitoring devices.

According to some embodiments, when an apnea event is predicted, the sensitivity of the monitor may be increased. As a non-limiting example, the threshold values for mitigation may be reduced.

According to some embodiments, a personalized learning process may be applied. According to some embodiments, the learning process may facilitate correlating between pre-apneic patterns and apnea events of the patient. Thus, since different patients may act slightly different, the learning process may increase the sensitivity of the monitor.

According to some embodiments, the system may further include a stimulating unit. The stimulating unit may be configured to provide a stimulation to the subject, whereby the stimulation is configured to provide mitigation of an apnea event (either prior to the event or concomitantly with the event). In some embodiments, the stimulation provided by the stimulating unit does not wake the subject (that is, the subject's sleep is not interrupted by the stimulation). In some embodiments, the stimulating unit may provide a tactile stimulation, such as a low threshold vibration, a low threshold electrical stimulation, and the like, capable of mitigating an apnea event, but which does not induce wakening of the subject. In such embodiments, the stimulating unit includes a vibration subunit capable of providing tactile stimulation to the subject. In some embodiments, the tactile stimulating unit is wearable on the subject body, for example, on a limb. In some embodiments the stimulating unit is configured to provide an audible stimulation, such as a low frequency sound wave, capable of mitigating an apnea event, but which does not induce wakening of the subject. In such embodiment, the stimulating unit includes a speaker capable of producing sound at a required frequency. In some embodiments, the stimulating unit may be wearable by the subject.

In further embodiments, the stimulating unit may be configured to provide/administer gas to the subject, thereby mitigating an apnea event, without waking the subject. In some embodiments, the stimulating unit is configured to provide/dispense/issue/administer doses of gas, which can mitigate an apnea event. In some embodiments, the gas is CO₂, whereby administration of said CO₂ to the patient can mitigate an apnea event, without wakening the subject. Stimulation by gas, such as CO₂, can induce breathing reflex in the subject, thereby mitigating the apnea event and restore breathing by provoking increase of respiratory drive. In some embodiments, the stimulating unit configured to provide gas to the subject may be wearable or in close proximity to the subject. For example, the stimulating unit may include a mask wearable on the subject face. For example, the stimulating unit may include a tube or cannula capable of providing/administering gas supply to the subject. In some embodiments, the stimulating unit includes a gas reservoir and/or a gas dispensing subunit (such as, for example, a pump). In some embodiments, the amount/level of the stimulation and/or the timing of the stimulation may be identical or different for each apnea event. In some embodiments, the level and/or timing of the stimulation may be determined by stimulating unit. In some embodiments, the level and/or timing of the stimulation may be determined (directly or indirectly) by the processing unit.

In some embodiments, the stimulating unit may be associated (directly or indirectly) with the processing unit. In some embodiments, the stimulating unit may be associated (directly or indirectly) and controlled by the processing unit. In some embodiments, the processing unit may control the timing and/or amount and/or type of stimulation provided by the stimulating unit to the subject. According to some embodiments, the same cannula may be used for both sampling and stimulation.

According to some embodiments, the measured physiological parameters may be determined or calculated over a period of time, in order to effectively predict apnea events over time. The period of time may be predetermined. In some embodiments, global features, which are characteristic of general apnea events may be determined and used for the prediction of an apnea event. In some embodiments, individual features (learnt by the monitor), which are characteristics of apnea events of a specific subject may be identified/ determined and used for the prediction of an apnea event in that subject.

According to some embodiments, the apnea event and prediction thereof may be determined by various calculations and algorithms and may be assigned a value of certainty of prediction. In some embodiments, the apnea prediction value may be calculated by various means, such as, for example, by use of mathematical equations, algorithms, formulas, pattern detection and the like, that may take into consideration the values or derivatives of the values of the physiological parameters measured by the one or more monitoring devices.

According to some embodiments the system may include a user interface that may allow a user to select the data to be displayed and to control various operating parameters and/or display stored historical data to provide enhanced predictability of apnea events by the system. Moreover, different displays may be included to accommodate different needs of the different users.

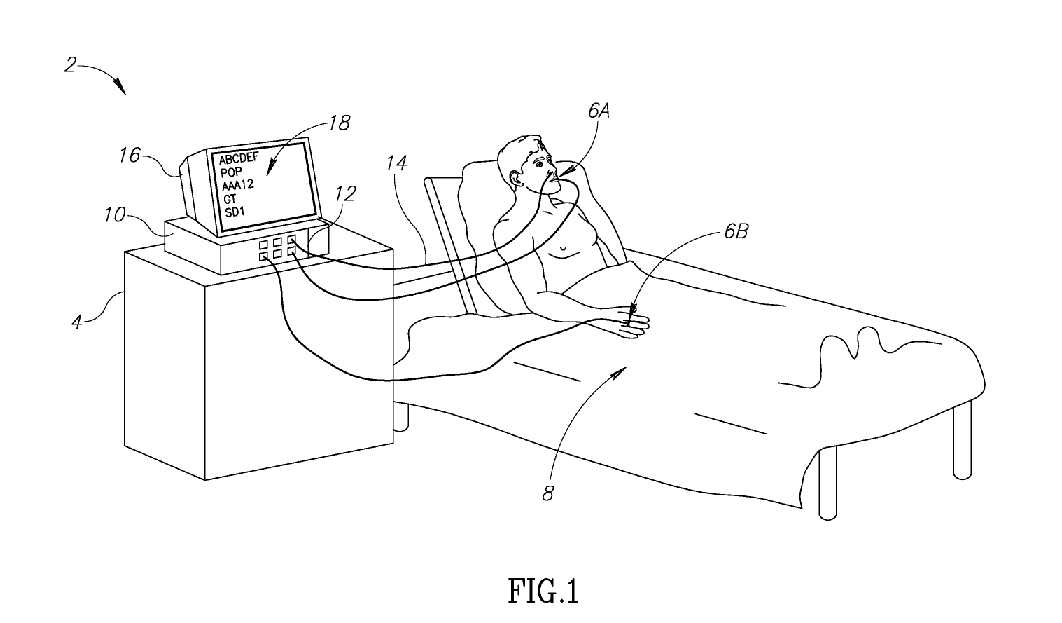

Reference is now made to FIG. 1, which is a schematic illustration of a system, according to some embodiments. As shown in FIG. 1, system (2) may include one or more medical monitoring devices (shown as one integrated device (4)). The medical monitoring devices may include, for example, but not limited to: a capnograph, a pulse oximeter, spirometer, a breath flow meter, heart rate sensors, blood pressure sensors, ECG, EEG, and the like. The monitoring devices include various sensors, such as, sensors 6A-B that may be configured to obtain/sense/measure various physiological parameters of subject (8). The exemplary sensors shown in FIG. 1 are CO₂ sensor (6A) and pulse oximetry sensor (6B). The one or more sensors may be connected directly or indirectly to the patient. The parameters thus measured/obtained/calculated may include, for example, such parameters as, but not limited to: EtCO₂, CO₂ concentration levels, CO₂ waveform pattern, CO₂ volume, SpO₂, heart rate, blood pressure, blood flow, and the like. System 2 may further include a processing unit, such as, for example, processing unit (processing logic, 10), that may be used to receive information from the sensors and to process the information to detect and/or predict apnea event, based on the measured parameters or data derived therefrom. The processing unit may include any type of firmware, hardware and/or software. The connection between the processing unit and the sensor(s) may include any type of communication route, such as, for example, use of wires, cables, wireless, and the like. The system may further include a stimulating unit (12) configured to provide stimulation to the subject, such that the apnea event is mitigated, without waking up or arousing the subject. The stimulation issued by the stimulating unit may be any a tactile stimulation, an audible stimulation and/or a stimulation in the form of a gas supplied to the subject. As exemplary shown in FIG. 1, the stimulation is in the form of a gas supplied to the subject, via tube (14), capable of providing gas, such as, CO₂, to the subject. The gas supplied/administered to the subject may be at varying doses/amount and the supply of which may be controlled by the stimulating unit, which in turn may be controlled by the processing unit. The system may further include one or more displays (such as, for example, display 16 in FIG. 1), that may be used to present the data collected and determined by the processing unit, the change over time of the data, predicted apnea event, the timing and/or quantity/amount/level of stimulation, and the like. In addition, the system may further include a user interface, such as, user interface 18 that may allow a user to input subject related data and/or control any of the operating parameters of the system. The system may further include data storing subunits, capable of storing data, such as, historical data, subject related data, measured parameters of data derived therefrom, and the like.

According to some embodiments, there is provided a method for predicting and optionally mitigating apnea event of a subject. In some embodiments, the method may include obtaining measurements of one or more physiological parameters of the subject, identifying/predicting apnea event, based on the measured parameters (or data obtained therefrom), and providing a stimulation to mitigate the apnea event.

According to some embodiments, there is provided a method for predicting and optionally mitigating apnea event of a subject. In some embodiments, the method may include obtaining measurements of one or more physiological parameters of the subject, identifying/predicting apnea event, based on the measured parameters (or data obtained therefrom), and providing a stimulation to mitigate the apnea event. In some embodiments, the physiological parameters, or data derived therefrom, may include such parameters as, for example, but not limited to: breath related parameters (as determined, for example, based on expired CO₂ (measurements of CO₂ in exhaled breath), airflow, respiration rate, respiration effort, and the like), heart related parameters (such as heart rate), blood related parameters (such as, blood pressure, oxygen saturation, blood flow), brain activity related parameters (such as brain electrical activity), and the like, or combinations thereof.

According to some embodiments, there is provided a method for predicting and optionally mitigating apnea event of a subject, the method may include obtaining measurements of expired CO₂; and one or more additional physiological parameters of subject; and identifying/predicting apnea event, based on the measured parameters (and/or data obtained therefrom), and providing a stimulation to mitigate the apnea event. According to some embodiments, the additional parameters (in addition to expired CO₂) may be, heart rate, airflow, SpO₂, respiratory rate, respiratory effort, blood pressure, brain activity, data derived therefrom, and the like.

In some exemplary embodiments, sleep apnea (such as obstructive sleep apnea) may be characterized by repetitive cycles of cessation of breathing, followed by rapid breaths, before cessation of breathing again. Various distinct repeatable changes in various physiological parameters or data derived therefrom during such cycles may be detected/determined and used to predict an upcoming apnea event.

In some embodiments, the method may further include manipulation and/or processing of the measured parameters, to generate data related to the measurements, prior to or simultaneously while being integrated for determination/prediction of apnea. In some embodiments, the measured physiological parameters may be determined or calculated over a period of time, in order to effectively predict apnea events over time. The period of time may be predetermined. In some embodiments, global features, which are characteristic of general apnea events may be determined and used for the prediction of an apnea event. In some embodiments, individual features, which are characteristics of apnea events of a specific subject may be identified/ determined and used for the prediction of an apnea event in that subject.

According to some embodiments, the apnea event and prediction thereof may be determined by various calculations and algorithms and may be assigned a value of certainty of prediction. In some embodiments, the apnea prediction value may be calculated by various means, such as, for example, by use of mathematical equations, algorithms, formulas, and the like, that may take into consideration the values or derivatives of the values of the physiological parameters measured by the one or more monitoring devices.

According to some embodiments, learning and optimization of determination and prediction of apnea events may be carried out using various methods, such as, for example, but not limited to: neural networks, a support vector machine (SVM), genetic algorithms, simulated annealing and expectation-maximization (EM), learning systems based on historic data, and the like.

In some exemplary embodiments, various CO₂ related parameters may be derived from the measurement of CO₂ in breath, such as, for example but not limited to: CO₂ waveform and parameters related thereto (such as, for example, but not limited to: EtCO2, changes in EtCO₂, a slope of the increase and/or decrease in the CO₂ concentration, a change in a slope of the increase and/or decrease in the CO₂ concentration, time to rise to a predetermined percentage of a maximum value of CO2 concentration, a change in time to rise to a predetermined percentage of a maximum value of CO₂ concentration, an angle of rise to a predetermined percentage of a maximum value of CO₂ concentration, a change in an angle of rise and/or fall to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, breath cycle, CO₂ concentration in expired air, and the like, or any combination thereof. Such parameters may be indicative of apnea event and may be used to predict an apnea event. For example, increase in breath rate, changes in breath depth, changes in CO2 waveform, cessation of breath, may be indicative and/or predictive of apnea event. According to some exemplary embodiments, tracking/tracing/reviewing CO₂ waveforms of the subject over time may be indicative of an upcoming apnea event. For example, in cases where a new waveform is not detected for a period of time (for example 10 seconds or any determined period of time), it may be indicative of an apnea event. Analysis of changes in CO₂ waveforms over time, can provide indicating signs that an apnea event is expected.

For example, in some embodiments, various parameters (data) derived directly or indirectly from pulse oximetry measurements may be obtained, such as, heart rate (HR, pulse rate), respiration effort (RE), amplitude of cardiac pulses (as determined based on the photoplethysmograph signal (PPG)); modulation of the amplitude of cardiac pulses, percent modulation (PMod) of the signal, changes in the PMod, changes in blood flow (for example, change in venous return flow from the peripherals), and the like, or combinations thereof.

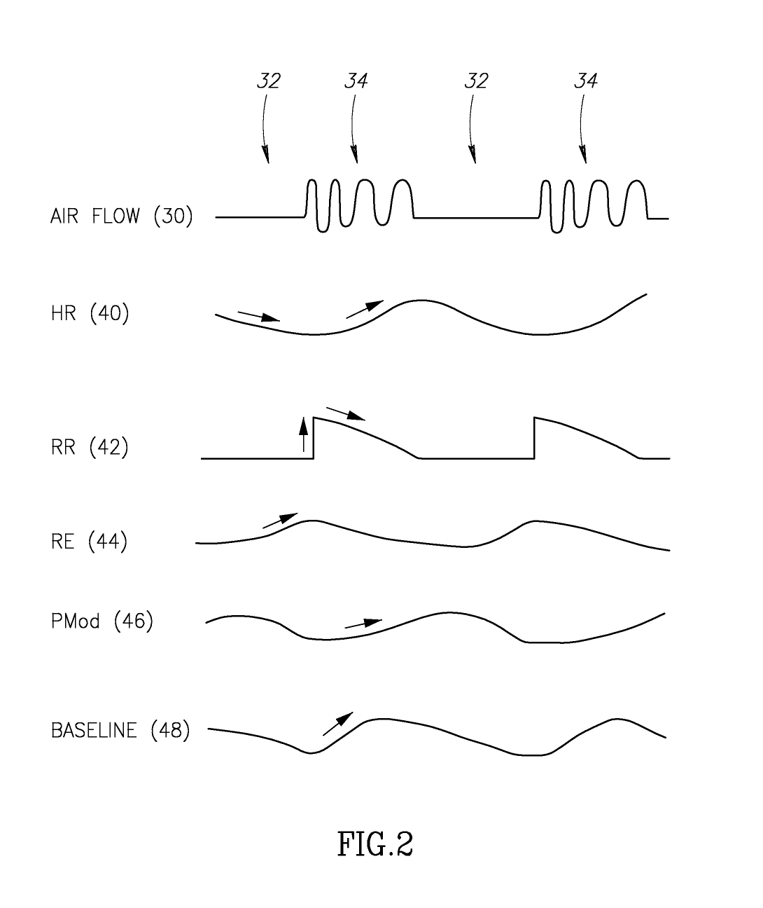

Reference is now made to FIG. 2, which depicts graphs of various physiological parameters prior to, during, and after apnea event, that can be used in a method for predicting and event. As shown in FIG. 2, airflow measurements (30) are shown to contain intervals of no flow (i.e. apnea (32)), interspersed by regions of two-way flow (i.e. breaths (34)). The heart rate (HR, 40) of the subject increases on the resumption of airflow, as the subject arouses and cardiac output increases, and reduces the cessation of airflow. In some exemplary embodiments, the pattern of the HR may be learned over several apnea cycles and used to predict the onset of the cessation of airflow. The respiration rate (RR, 42), begins at a fast rate on the resumption of airflow, slowing down thereafter until the apnea event. In some exemplary embodiments, the pattern of the RR may be learned over several apnea cycles and used to predict the onset of the cessation of airflow. The respiration effort (RE, 44) of the patient may be determined from the amplitude of modulations in the waveform of the plethysmograph. This measures the respiratory drive of the subject. In some embodiments, the respiration effort displays a characteristic behavior during the apnea cycle, whereby it increases just prior to the resumption of airflow, where the subject is beginning to arouse and attempting to take in air, this becomes stronger (as the subject is breathing against a closed airway), peaking just as the airway opens and airflow resumes. The vasoconstriction of the subject may be determined based on the percent modulation (pMod, 46) parameter (i.e., the amplitude of the pulsatile component of the plethysmograph waveform. In some embodiments, the pMod displays a characteristic behavior during the apnea cycle whereby it reduced to a minimum around the time of airflow resumption as the patient vasoconstricts as part of the arousal process. The change in venous return from the peripherals may be determined from the baseline of the plethysmograph waveform (baseline, 48). In some embodiments, the baseline displays a characteristics behavior during the apnea cycle whereby it reduces to a minimum just before the resumption of airflow, as the blood begins to pool in the peripherals. Additionally, in accordance with some embodiments, when apnea event is occurring, a pattern of rapid breathing may be identified based on, for example, changes to the CO₂ waveform and frequency of the breath cycles, which can further be used for the prediction of an apnea event.

According to some embodiments, one or more of the following exemplary parameters (each indicative/predictive by itself of an apnea event) may be combined in order to provide a more accurate prediction of the onset of an apnea event: the HR-based airflow prediction parameter, the RR-based airflow prediction parameter, the RE-based airflow parameter, the pMod-based airflow prediction parameter, the baseline-based airflow prediction parameter, the CO₂ related parameters (such as changes in CO₂ waveform, frequency of breath cycles), and the like. In some embodiments, one or more of the above mentioned exemplary parameters may be combined with additional physiological parameters, such as, but not limited to: changes in blood pressure, changes in EEG signal, regional saturation changes, changes in pulse transit time, and the like.

According to some embodiments, in some cases, after detection of apnea events over time, an apnea cycle time may be estimated and may further be used for the prediction of the time point of the onset of the next apnea event.

According to some embodiments, there is provided a method used in a system for predicting and mitigating an apnea event, the system may include one or more medical monitoring devices configured to measure one or more physiological parameters of a subject; a processing unit capable of integrating the measured physiological parameters to determine and predict an apnea event of the subject, based on the measured parameters; and a stimulating unit configured to provide a stimulation to the subject that can mitigate the apnea event. According to some embodiments, the physiological parameters may be such parameters as, but not limited to: expired CO2, CO2 waveforms, airflow, respiration rate, respiration effort, heart rate, SpO₂, blood pressure, brain activity, data derived therefrom or related the

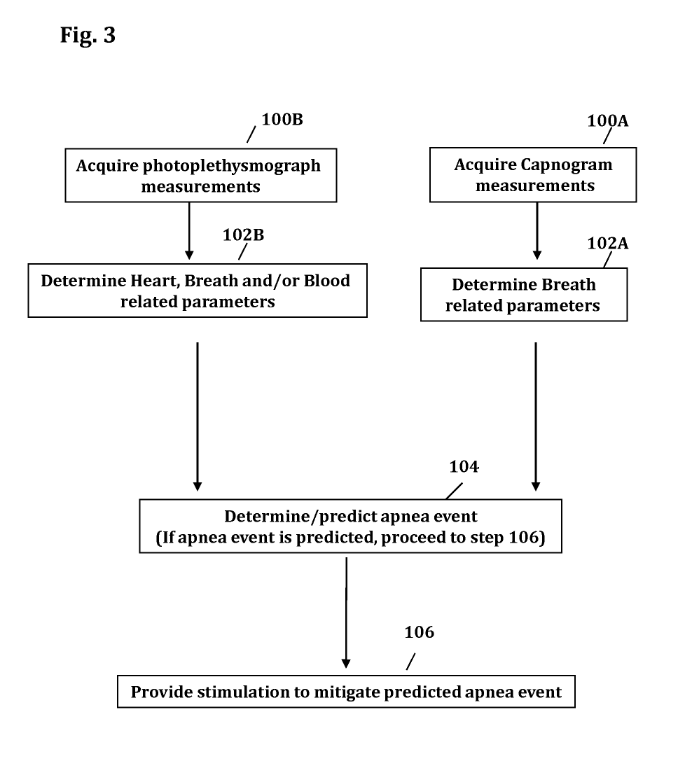

Reference is now made to FIG. 3, which is a schematic block diagram of steps in a method for predicting and mitigating an apnea event of a subject and/or for mitigating arousal of the subject, according to some embodiments. As shown in FIG. 3, measurements of one or more physiological parameters of a subject are obtained from the appropriate corresponding monitoring devices. In the example shown in FIG. 3, at step 100A, capnograph measurements are obtained, and at step 100B, photoplethysmograph (pulse oximetry) measurements are obtained. Each of steps 100A-B may be performed simultaneously, sequentially or independently of each other. Next, the obtained measurements may be further proceed or analyzed to determine various related parameters. For example, at step 102A, various breath related parameters (such as, for example, airflow, respiration rate, expired CO₂ related parameters) are determined, based on the capnograph measurements. Exemplary CO₂ related parameters include such parameters as, but not limited to: CO₂ waveform and parameters related thereto (such as, for example, but not limited to: EtCO₂, changes in EtCO₂, a slope of the increase in the CO₂ concentration, a change in a slope of the increase and/or decrease in the CO₂ concentration, time to rise and/or fall to a predetermined percentage of a maximum value of CO₂ concentration, a change in time to rise and/or fall to a predetermined percentage of a maximum value of CO₂ concentration, an angle of rise and/or fall to a predetermined percentage of a maximum value of CO₂ concentration, a change in an angle of rise and/or fall to a predetermined percentage of a maximum value of CO₂ concentration, breath to breath correlation, a change in breath to breath correlation, a CO₂ duty cycle, a change in CO₂ duty cycle, minute ventilation, a change in minute ventilation or any combination thereof), respiration rate, respiration effort, breath cycle, CO₂ concentration in expired air, and the like, or any combination thereof. For example, at step 102B, various breath, heart and/or blood related parameters are determined, based on the pulse oximetry measurements. Such exemplary parameters may be such parameters as, but not limited to: heart rate (pulse rate), respiration rate, respiration effort, amplitude of cardiac pulses; modulation of the amplitude of cardiac pulses, percent modulation (PMod) of the signal, vasoconstriction, changes in venous blood return from peripheries, and the like, and combinations thereof. Each of steps 102A-B may be performed simultaneously, sequentially or independently of each other. Next, at step 104, the various parameters that have been determined in steps 102A-B may be integrated (for example, by a processing unit) to determine, identify and/or predict apnea event. Further in step 104, if based on the provided measurements (or data derived therefrom), an apnea event determined to occur (i.e., an apnea event is predicted), stimulation is provided in step 106. The stimulation provided in step 106 may be provided by a stimulating unit. The stimulation provided in step 106 is configured to mitigate the apnea event, without affecting arousal of the subject. The stimulation provided in step 106 may be any type of stimulation, such as, for example, audible stimulation, tactile stimulation, stimulation of the nervous system (such as the autonomic nervous system), stimulation of the respiration regulatory center, stimulation of respiratory system, and the like, or combinations thereof. For example, tactile stimulation may be provided in the form of an electric pule, capable of causing mitigation of an apnea event. For example, stimulation of the respiration system (or nervous system) may be provided in the form of providing gas (such as CO₂) to the patient, to stimulate the respiratory system and mitigate the apnea event.

According to some embodiments, the systems and methods disclosed herein provide for a non-invasive prediction and mitigation of apnea event.

According to some embodiments, the systems and methods disclosed herein provide for prediction and mitigation of an apnea event, without affecting the awareness of the subject suffering from apnea, that is, without waking the subject while preventing/mitigating the apnea event.

It is understood by the skilled in the art that the processor of the system is configured to implement the method as essentially described herein.

In the description and claims of the application, each of the words “comprise” “include” and “have”, and forms thereof, are not necessarily limited to members in a list with which the words may be associated.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

### EXAMPLES

### Example 1

### Prediction and Mitigation of Sleep Apnea Based on Measurments of Physiological Parameters of a Subject

Apnea events of a test subject are determined and predicted based on combination of measurements of the following physiological parameter: expired CO₂, airflow, respiratory rate (RR), respiration effort (RE) (as determined by capnograph measurements), heart rate, pMod (percent modulation of plethysmograph signal).

The sleeping subject is continuously being monitored by a capnograph and a pulse oximeter. When apnea event is about to occur, changes to the CO₂ waveform, and frequency of the breath cycles, are identified based on the capnography signal. Additionally, changes in heart rate, respiration effort and PMod (calculated by dividing the amplitude of the cardiac pulses (obtained based on the photoplethysmograph signal) by the baseline of the signal), are observed. The integrated, combined analysis of the identified changes in the measured parameters and the data derived therefrom, provide a reliable prediction of an apnea event. Consequently, stimulation is provided to the subject, in the form of CO₂ administration. The concentration of CO₂ administered may be determined based on the measured EtCO₂, so as to mitigate the apnea event, without arousing the test subject. For example, the concentration supplied may be similar to the measured EtCO₂ or higher than the measured EtCO₂ higher (e.g. slightly higher such as 0.5-10% higher than the measured EtCO₂). According to some embodiments, the amount of CO₂ administered may be required to be significantly higher than the measured EtCO₂ (e.g. more than 10% higher than the measured EtCO₂).

The examples described above are non-limiting examples and are not intended to limit the scope of the disclosure. The described examples may comprise different features, not all of which are required in all embodiments of the disclosure.

What is claimed is:

1. A system for predicting and mitigating sleep apnea event in a subject, the system comprising:
one or more medical monitoring devices configured to measure one or more physiological parameters of the subject;
a processing unit configured to integrate the one or more physiological parameters of the subject, to predict an apnea event, based on the measured parameters; and
a stimulating unit configured to provide a stimulation to the subject, prior to the apnea event, thereby mitigating said apnea event.

2. The system of claim 1, wherein said processing unit is further configured to determine a patient specific pre-apneic pattern in the one or more physiological parameters by applying a learning algorithm to the measured one or more physiological parameters.

3. The system of claim 2, wherein predicting the apnea event comprises detecting the determined patient specific pre-apneic pattern in the one or more measured physiological parameters.

4. The system of claim 1, wherein the medical monitoring device comprises a capnograph or a pulse oximeter.

5. The system of claim 4, wherein the medical monitoring device further comprises: capnograph, pulse oximeter, breath flow sensor, Electrocardiogram (ECG), Brain activity monitoring device, or combinations thereof.

6. The system of claim 1, wherein the physiological parameters of the subject comprises: breath related parameters, heart related parameters, blood related parameters, brain electrical activity, parameters derived therefrom, or combinations thereof.

7. The system of claim 6, wherein the breath related parameters comprises airflow, respiration rate, respiration effort, breath flow, expired CO₂, or combinations thereof.

8. The system of claim 7, wherein the expired CO₂ related parameters comprises: CO₂ concentration in breath, EtCO₂, CO₂ waveform, and combinations thereof.

9. The system of claim 6, wherein the heart related parameters comprises heart rate, amplitude of cardiac pulses, Percent Modulation (PMod) of the cardiac pulses, or combinations thereof.

10. The system of claim 6, wherein the blood related parameters comprises blood pressure, Oxygen saturation (SpO₂), blood flow, or combinations thereof.

11. The system of claim 1, wherein the stimulating unit does not induce wakening or arousing the subject.

12. The system of claim 1, wherein the stimulating unit comprises a gas emitting unit, a gas dispenser, a tactile unit, an audible unit, or combinations thereof.

13. The system of claim 1, further comprising a display unit configured to display one or more of: one or more of the measured parameters, parameters derived from the measured parameters, an indication for a predicated apnea event, an indication of the activation of the stimulating unit, or combinations thereof.

14. A method for predicting and mitigating sleep apnea event in a subject, the system comprising:
a. obtaining a measurement of one or more physiological parameters of the subject;
b. predicting an apnea event based on the measured parameters of the subject; and
c. issuing a stimulation prior to the apnea event to mitigate said apnea event, without awakening the subject.

15. The method of claim 14, further comprising determining a patient specific pre-apneic pattern in the one or more physiological parameters by applying a learning algorithm to the measured one or more physiological parameters.

16. The method of claim 15, wherein predicting the apnea event comprises detecting the determined patient specific pre-apneic pattern in the one or more measured physiological parameters.

17. The method of claim 14, wherein the one or more physiological parameter is a breath related parameter.

18. The method of claim 17, wherein the breath related parameters comprises airflow, expired CO₂, respiration effort, breath flow, respiration rate, or combinations thereof.

19. The method of claim 18, wherein the expired CO₂ related parameters comprises: CO₂ concentration in breath, EtCO₂, CO₂ waveform, or combinations thereof.

20. The method of claim 14, wherein the stimulation comprises a gas provided to the subject, an audible stimulation, a tactile stimulation, or combinations thereof.
